(19) United States
(12) Patent Application Publication
(10) Pub. No.: US 2020/0113501 A1
(43) Pub. Date: Apr. 16, 2020

(54) Automated Data Acquisition, Appraisal and Analysis in Noninvasive Rapid Screening of Neuro-Otologic Conditions Using Combination of Subject's Objective Oculomotor Vestibular and Reaction Time Analytic Variables

(71) Applicant: Neuro Kinetics, Inc., Pittsburgh, PA (US)

(72) Inventors: Alexander D. Kiderman, Pittsburgh, PA (US); Ian Shirey, Pittsburgh, PA (US)

(21) Appl. No.: 16/599,556

(22) Filed: Oct. 11, 2019

Related U.S. Application Data
(63) Continuation of application No. 15/233,845, filed on Aug. 10, 2016, now abandoned.
(60) Provisional application No. 62/203,131, filed on Aug. 10, 2015.

(51) Int. Cl. A61B 5/16; A61B 5/00; A61B 5/12; A61B 3/00; A61B 3/113

(52) U.S. Cl. CPC A61B 5/162; A61B 5/4076; A61B 5/4064; A61B 3/0025; A61B 5/7282; A61B 3/0041; A61B 3/113; A61B 5/12

(57) ABSTRACT

An automated data acquisition, appraisal and analysis in noninvasive rapid screening of neuro-otologic condition using combination of subject's objective oculomotor, vestibular and reaction time analytic variables, comprises the steps of: establishing an oculomotor, vestibular and reaction time testing protocol for specific neuro-otologic condition which protocol included eye tracking aspects and which protocol obtains a combination of a subject's objective oculomotor, vestibular and reaction time analytic variables; coupling an eye tracker to subject; automatically establishing a region of interest for the eye tracker in which the region of interest is centered on the subject's pupils; conducting the oculomotor, vestibular and reaction time testing protocol using pre-programmed/automated voice instructions to the subject to obtain a combination of a subject's objective oculomotor, vestibular and reaction time analytic variables; and calculating and displaying a normalized composite test protocol score (0-1) on graphical progressive background.

### RELATED APPLICATIONS

The present application is a continuation of U.S. patent application Ser. No. 15/233,845 filed Aug. 10, 2016, entitled “Automated Data Acquisition, Appraisal and Analysis in Noninvasive Rapid Screening of Neuro-Otologic Conditions Using Combination of Subject's Objective Oculomotor, Vestibular and Reaction Time Analytic Variables”.

This application claims the benefit of U.S. patent application Ser. No. 62/203,131 filed Aug. 10, 2015 entitled “Automated Data Acquisition, Appraisal and Analysis in Noninvasive Rapid Screening of Mild Traumatic Brain Injury using Combination of Subject's Objective Oculomotor, Vestibular and Reaction Time Analytic Variables.”

### BACKGROUND OF THE INVENTION

### 1. Field of the Invention

The present invention relates to noninvasive rapid screening of neuro-otologic conditions, such as Traumatic Brain Injury (TBI including mild Traumatic Brain Injury or mTBI), and psychological health, and more specifically to quantitative, non-invasive, clinical diagnosis of traumatic brain injury. The present invention is directed to noninvasive rapid screening of mild traumatic brain injury using combination of subject's objective oculomotor, vestibular and reaction time analytic variables.

### 2. Background Information

Traumatic Brain Injury (TBI) or concussions are a common occurrence in both military operations and training, and in the general population, e.g. sports concussions. Traumatic Brain Injury (TBI) is the result of a blunt blow, jolt or blast overpressure to the head that disrupts brain function. The subset of mild TBI, or mTBI, has represented a harder segment of TBI to diagnose. Within this application mTBI is a subset of TBI. The terms mild TBI (mTBI) and concussion are commonly used interchangeably in the art, and have been linked with Post Traumatic Stress Disorder. The severity of head injuries range from a brief change in mental status or consciousness to extended unconsciousness and amnesia. In severe or multiple concussion cases, personality changes can occur with devastating results.

The Centers for Disease Control and Prevention has noted that TBI “is a major cause of death and disability in the United States, contributing to about 30% of all injury deaths. Every day, 138 people in the United States die from injuries that include TBI. Those who survive a TBI can face effects lasting a few days to disabilities which may last the rest of their lives. Effects of TBI can include impaired thinking or memory, movement, sensation (e.g., vision or hearing), or emotional functioning (e.g., personality changes, depression). These issues not only affect individuals but can have lasting effects on families and communities.” Additionally they have estimated that at least 3.17 million Americans currently have a long-term or lifelong need for help to perform activities of daily living as a result of a TBI. Currently there is no accepted clinical method to objectively detect mTBI. The Center for Disease Control (at httb://www.cdc.gov/TraumaticBrainInjury/statistics.html) estimates that “About 75% of TBIs that occur each year are concussions or other forms of mild TBI.” For further background please see Brain Injury Association of America at www.BIAUSA.org as The Brain injury Association of America (BIAA) is the country's oldest and largest nationwide brain injury advocacy organization.

Military personnel, despite using strong protective devices, frequently suffer blast injuries to the head. In a study conducted at the Walter Reed Army Medical Center, 62% of Operation Iraqi Freedom combat wounded troops showed symptoms of mild to severe brain injuries, and of these, 91.6% had possibly sustained a TBI injury as a result of a blast. A number of recent studies have substantiated the presence of vestibular deficits in the acute period following TBI. The Defense and Veterans Brain Injury Center (www.dvbic.org) is a part of the U.S. Military Health System, specifically, it is the traumatic brain injury (TBI) operational component of the Defense Centers of Excellence for Psychological Health and Traumatic Brain Injury (DCoE) founded in 1992 by Congress, and represent a source for further detailed background and state of the art for TBI and the effect on the military.

Proper treatment of TBI injury requires an accurate diagnosis of the structures affected. Proper treatment of TBI injury requires an accurate diagnosis of the structures affected. The mechanisms of injury in TBI cause a variety of abnormalities in the peripheral vestibular mechanisms, central vestibular structures, ocular-motor tracts, cerebellum, as well as all portions of the brain communicating with these structures. The onset of vestibular deficits generally occurs within seven to ten days post injury. While reported symptoms of dizziness resolve after three months, 15% have persistent symptoms one year later.

The foremost challenges in treating concussions are objectively diagnosing the presence of a concussive injury, judging its severity, and choosing an appropriate therapeutic course. Currently, essentially no technology exists that has demonstrated sufficient sensitivities and specificities to be cleared by the FDA as a general aid in the diagnosis of concussion. This situation provides clinicians with restrictive and or subjective options for concussion assessment and management. Imaging modalities, such as MRI or diffusion tensor imaging (DTI), are costly, not portable, have a large footprint, and (in the case of MRI) typically show abnormalities only when there is significant structural damage present, i.e. a moderate or severe TBI. While computed tomography scans are comparatively less costly and are well-suited to detecting structural abnormalities, e.g. hematomas, they provide little insight into the fine-scale axonal and metabolic dysfunctions that characterize concussion. Neuro-psychological tests such as MACE™ or ImPACT™, are portable, but are subjective and have sensitivities and specificities that are insufficiently robust for reliable diagnostic classification.

It has been proposed to employ easily administered and objective tests of oculomotor, vestibular, and reaction time (OVRT) functions as metrics of neurological performance across a broad range of neuro-functional and neuro-anatomical domains. For reference please see U.S. Published Patent Applications 2015-0335278, 2015-0018709, 2014-0327880, 2014-0192326, and 2010-0094161, which published patent applications are incorporated herein by reference.

It is the object of the present invention to address the deficiencies of the prior art to provide robust clinical OVRT testing as a concussion diagnostic tool, or other specific neuro-otologic condition diagnostic tool.

### SUMMARY OF THE INVENTION

The present invention is drawn to the development of Automated Data Acquisition, Appraisal and Analysis systems and methods employing easily administered and objective tests of oculomotor, vestibular, and reaction time (OVRT) functions as metrics of neurological performance across a broad range of neuro-functional and neuro-anatomical domains. This invention provides robust a clinical OVRT testing tool for use as a concussion or mTBI (or TBI) diagnostic tool.

One aspect of the present invention provides an automated data acquisition, appraisal and analysis in noninvasive rapid screening of neuro-otologic condition using combination of subject's objective oculomotor, vestibular and reaction time analytic variables, comprising the steps of: establishing an OVRT testing protocol for specific neuro-otologic condition which protocol included eye tracking aspects; coupling an eye tracker is coupled to subject; automatically establishing a region of interest for the eye tracker centered on the subject's pupils; conducting the OVRT testing protocol using pre-programmed/ automated voice instructions to the subject; confirming that each test is performed within preset parameters including automated eye image evaluation and advising the clinician of testing which is outside of present parameters; and displaying a normalized composite test protocol score (0-1) on graphical progressive background.

One aspect of the invention provides a method for detecting and scoring concussions which demonstrates both high sensitivity and specificity for concussion/mild traumatic brain injury (mTBI) detection and which employs easily administered and objective tests of 2015-0335278, 2015-0018709, 2014-0327880, 2014-0192326, and 2010-0094161 (OVRT) functions as metrics of neurological performance across a broad range of neuro-functional and neuro-anatomical domains.

These and other advantages are described in the brief description of the preferred embodiments in which like reference numeral represent like elements throughout.

### BRIEF DESCRIPTION OF THE FIGURES

FIG. 1 is a schematic flow chart of an automated data acquisition, appraisal and analysis in noninvasive rapid screening of specific neuro-otologic condition, such as mild traumatic brain injury, using combination of subject's objective oculomotor, vestibular and reaction time analytic variables according to the present invention;

FIG. 2 is a schematic view of an automatic establishment of a region of interest (roi) for the eye tracker centered on the subject's pupils within the noninvasive rapid screening of specific neuro-otologic condition according to the present invention; and

FIG. 3 is a schematic view of a display of a normalized composite test protocol score on a graphical progressive background of the noninvasive rapid screening of specific neuro-otologic condition according to the present invention.

### DESCRIPTION OF THE PREFERRED EMBODIMENTS

It is noted that, as used in this specification and the appended claims, the singular forms “a,” “an,” and “the” include plural referents unless expressly and unequivocally limited to one referent. It has been proposed to employ easily administered and objective tests of oculomotor, vestibular, and reaction time (OVRT) functions as metrics of neurological performance across a broad range of neuro-functional and neuro-anatomical domains. For reference please see U.S. Published Patent Applications 2015-0335278, 2015-0018709, 2014-0327880, 2014-0192326, and 2010-0094161, which published patent applications are incorporated herein by reference.

These prior patent applications and associated previous studies by the applicant, Neuro Kinetics, Inc. [NKI], and others have shown that various OVRT metrics are measurably altered following a concussion. NKI's prior works provided evidence that when using a battery of these tests with sensitive technologies, these detectable differences can be used to build a diagnostic model to separate concussion from control patients with high sensitivity and high specificity. NKI's prior works related to using a battery of these tests suggested that i) no single test included in the protocol in isolation could be used to dependably classify subjects into the concussion versus control groups, and ii) when key variables of multiple tests were weighted and combined (e.g. with a logistic regression model), classification success was very reliable.

The proposed models are built on a foundation of a battery of tests historically used in vestibular testing, neurotologic testing, and some used in neurologic diagnosis. These tests assess neurologic and vestibular functions by delivering stimuli to subjects and measuring their evoked reflexes and responses, observed primarily through eye movements. These eye movement responses are captured using an eye tracker such as a high speed, high resolution video technology (video-oculography or VOG) to measure eye movements and pupillary responses. Motion stimuli may be delivered by rotation of a specialized nystagmograph chair testing of functions such as the vestibular-ocular reflex (VOR). Visual stimuli are presented either within a goggle system, or with a laser projection or with full-field irregular illumination. Often this visual stimulation is testing a patient's ability to generate saccades (rapid re-targeting eye movements), smooth pursuit tracking of moving targets, or full-field movement-induced eye responses (optokinetic nystagmus, OKN). Reaction time data (both eye movement and physical non-reflexive responses such as button presses) are also collected in response to either visual or auditory stimuli. One acceptable OVRT test battery developed by the applicant is known as the NKI NOTC test battery and the NKI NOTC test battery is a comprehensive mix ranging from those assessing reflexive function (e.g. the VOR), to tests containing more cognitive or executive aspects, such as the anti-saccades test (where subjects must volitionally look away from a stimulus with a sudden onset). For OVRT testing to be widely used clinically, rigorous validation of its utility needs to be established.

A hypothesized physiological explanation for success of such models is that the multiple neural pathways responsible for performing the multiple OVRT functions being stimulated by OVRT testing captures functionality of a comprehensively broad array of brain structures. Eye neurons are found throughout the brain supporting the premise that a multitude of functional brain areas impact the production of the eye's precisely timed and highly coordinated movements and the precision or variability of reaction time responses. This further suggests that even subtle heterogeneous damage caused by a concussion to any of a wide range of brain areas can create oculomotor disturbances and reaction time disruptions, particularly within tasks that expose and tax the limitations of human perceptual and motor performance. Lastly, this implies that more than one test, in fact a battery of tests (two or more), is necessary to detect a concussion. OVRT testing appears to be an efficient, comprehensive and successful way to test neurologic function.

Importantly, the effects of brain injury on eye reflexes and responses to OVRT testing are not always detectable by unaided human clinicians, sensitive eye tracking technology is required to capture and quantify subtle differences in eye velocity, latency, smoothness, and precision.

Based on these principles and, as mentioned, NKI's review of the related published works, NKI chose to add a certain new tests to its existing OVRT battery of tests for its investigational protocols. Subsequently, it completed two exploratory clinical trials, in conjunction with Allegheny Singer Research Institute (ASRI) and Allegheny General Hospital (AGH), under the West Penn Allegheny Health System Institutional Review Board (IRB) titled, “Vestibular and Neuro-otologic Testing of Athletes with Concussions.” The protocol for these trials included 12 OVRT tests using NKI's video nystagmography (VNG) system (but no motion or rotation tests). Both trials tested high school athletes (ages 13 to 18) who had sustained a clinically diagnosed concussion (n=60, combined for the two studies) and high school athlete controls (n=265).

NKI and others are currently validating OVRT classification models or test protocols, which includes classifying subjects as suffering the effects of a concussion versus unimpaired controls using only OVRT tests, with a hypothesized sensitivity and specificity for concussion detection. The models created in these studies generate receiver operating characteristic (ROC) curves of greater than 0.9 AUC and do so consistently. The classification models being developed will result in the identification of the key separating variables and their optimized weighting to account for dependability, commercial clinic efficiencies and the necessary statistical sensitivities and specificities for a successful classification model, or composite concussion score. In general the classification models begin with a statistical review of subject data from a known population to identify potential key variables. Once the key variables that will make up the multi-variate model have been selected, the data will be reanalyzed, final validation trial parameters set, and the validation trial can then be run at clinical trial sites to confirm and validate the models. Steps may be rerun to optimize or to validate the model.

The establishment of specific multi-variate OVRT models or test protocols and specific key separating variables and their optimized weighting are outside the scope of the present application, other than the present invention will operate with any such given model.

The present invention is directed to the implementation of automated data acquisition and analysis for OVRT testing. One requirement for functional clinical utility is the ability of a system to produce repeatable, reliable results. For OVRT testing this necessitates the acquisition of objective, reliable data that minimizes subjective intervention on the part of the clinician. The present invention addresses this challenge by automating oculomotor data acquisition, signal quality assessment, and analysis for OVRT-testing.

The Technological Testing Platform component or aspect of the invention is the hardware and the software necessary for reliable acquisition and analysis. For hardware, the present invention uses an eye tracking system in which controlled stimulus of a variety of distinct testing can be selectively presented to the subject. Effectively the present invention uses the I-Portal® neuro-otologic testing center (NOTC) as the base on which the present invention may be easily and effectively implemented. This platform is a mature platform with a long history of operation and clinical utility. This lessens the risk inherent in a new indication, in this case the OVRT battery being tested with regard to concussions. For the software, the new OVRT concussion testing described herein effectively expands on the base system such and NKI's existing software platforms of VEST™ and I-Portal® brands.

The OVRT concussion protocol will typically use existing cleared tests with few additions of new tests. The existing tests have the advantage that they are already widely used in clinical practice for vestibular diagnostics and the acquisition and analyses for these tests have been implemented and verified in multiple studies and trials. Adding new tests are simple relative to the platform but require some training and validation. The adding of new tests is generally based on a review of the literature, to improve the outcomes with regard to concussions, and the acquisition and analyses for these new tests must be independently validated as part of the composite concussion score.

The present invention provides for automated appraisal and validation of eye movement signals; automated detection and removal of artifacts; and automated analysis of test metrics (latency, accuracy, gain relative to stimulus, etc.) with minimal operator intervention. The present invention will, operating with a given model for concussions or TBI, generate a composite score that reflects the probability of a subject having a concussion, i.e. a concussion score.

The present invention enhances acquisition software and simplifies its use as an important step in minimizing the influence of operator variability, and, hence, the reliability and repeatability of the platform. The implementation of automated analysis, which removes this variability, represents a significant advancement in the use of OVRT testing for neural dysfunction. Whereas other related versions of VNG technologies (for vestibular testing) require clinician adjustment at multiple stages of acquisition and analysis, the present invention effectively eliminates subjective manipulation of acquisition and analysis parameters. This development is viewed as critical for successful field deployment.

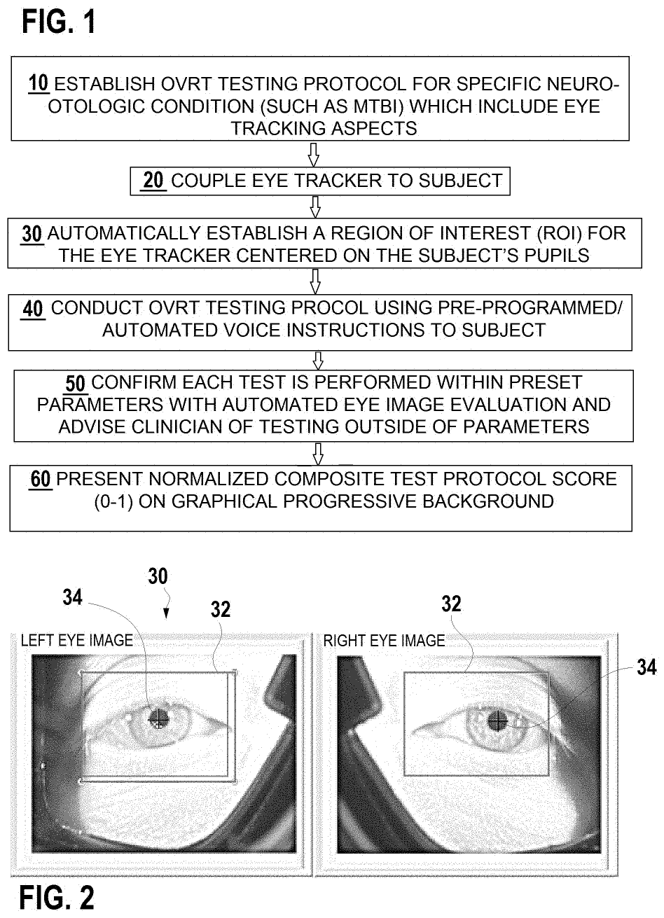

Implementation of automated analysis of the present invention includes three components set forth in greater detail below: Eye Tracking Aspects, Data Analysis Aspects, and User Interface Aspects. FIG. 1 is a schematic flow chart of an automated data acquisition, appraisal and analysis in noninvasive rapid screening of specific neuro-otologic condition, such as mild traumatic brain injury, using combination of subject's objective oculomotor, vestibular and reaction time analytic variables according to the present invention. Specifically at step 10 the present invention requires the establishment of a suitable OVRT testing protocol for specific neuro-otologic condition (such as mTBI) which protocol included eye tracking aspects. Next at step 20 a suitable eye tracker is coupled to subject. The present invention 30 automatically establishes a region of interest (roi) for the eye tracker centered on the subject's pupils. Then at step 40 the OVRT testing protocol is conducted using pre-programmed/automated voice instructions to the subject. The present invention, at step 50 confirms that each test is performed within preset parameters including automated eye image evaluation and advising the clinician of testing which is outside of present parameters. Finally the present invention, at step 60 displays a normalized composite test protocol score (0-1) on graphical progressive background.

The Eye Tracking Aspects of the invention includes three elements, namely a) Automate the setup process (including: region of interest [ROI] positioning, pupil centering, threshold setting); b) create pre-programmed/ automated voice instructions for each test; and c) automate confirmation of correct eye tracking settings.

The Automated setup in the eye tracking aspects will decrease setup time and create an interface usable by a technician or medic with minimal training which decreases opportunities for user errors.

As shown in FIG. 2 the invention, these parameters for the region of interest (ROI) 32 are set using an automatic threshold, and fixed with the ROI 32 self-positioning on pupil center 34. Precise calculation of pupil position and area requires a high contrast image of the eye, i.e. an image in which each pixel has been reduced to black or white. To translate the acquired grayscale eye image into this binary format, a threshold gray value must be set (all pixels below that value become black, the remaining become white). In existing technologies, the operator must constantly monitor the threshold in the two eye images, and instantaneously adjust as necessary. In the present invention, the threshold is automatically optimized for each frame of eye image data. Automatic threshold and ROI 34 are potentially more reliable than human adjustment and certainly decrease variability between operators.

Further, using pre-programmed/ automated voice instructions for each test removes variability between the tests due to clinician/technician error, and minimizes error due to subject misconception of testing protocol 40.

Adding an automated confirmation of correct eye tracking settings provides a simple automated assurance that the specific tests are being performed within the desired settings again minimizes erroneous results due to clinician/technician error. Feedback to the clinician/technician when confirmation is not observed is provided to further facilitate the testing protocol. Note that the correct eye tracking settings are part of the particular models and of the tests within the given models. The present invention is adding the step of confirming that each section of the battery of tests is maintained within the designated testing parameters.

The Data Analysis Aspects of the present invention further include i) an Automated eye image evaluation (e.g., blink detection/rejection/interpolation), filter settings and analysis of concussion tests; and ii) confirmation of acceptable test execution.

Streamlining the data analysis will decrease testing time, create an interface that can be used by an inexperienced technician or medic with minimal training, and limit opportunities for clinician/technician errors. Currently, it is up to the clinician to examine eye position data and eliminate samples in which the subject's eye position was not recorded properly, e.g. due to the eyelid temporarily occluding the eye, or improper selection of eye image thresholds as addressed above. In the present invention, the invalid data is automatically identified and removed. The implementation of this aspect of the present invention depends upon certain features that identify eye data signals of poor quality, e.g. outright failures to acquire the pupil (gaps in the data), fast oscillations indicating an unstable threshold, and changes in position or velocity that are not physiologically possible for eye movements. The quantifying these features is first and these quantified values are compared with thresholds such that the system can remove anomalies (those above/below/outside of given thresholds) from the data stream, and in cases where there is insufficient valid data remaining, the operator can be alerted that the test must be run again. Thus the system will check the remaining data points within a testing protocol with given thresholds to assure there is actual meaningful results after anomalies have been removed.

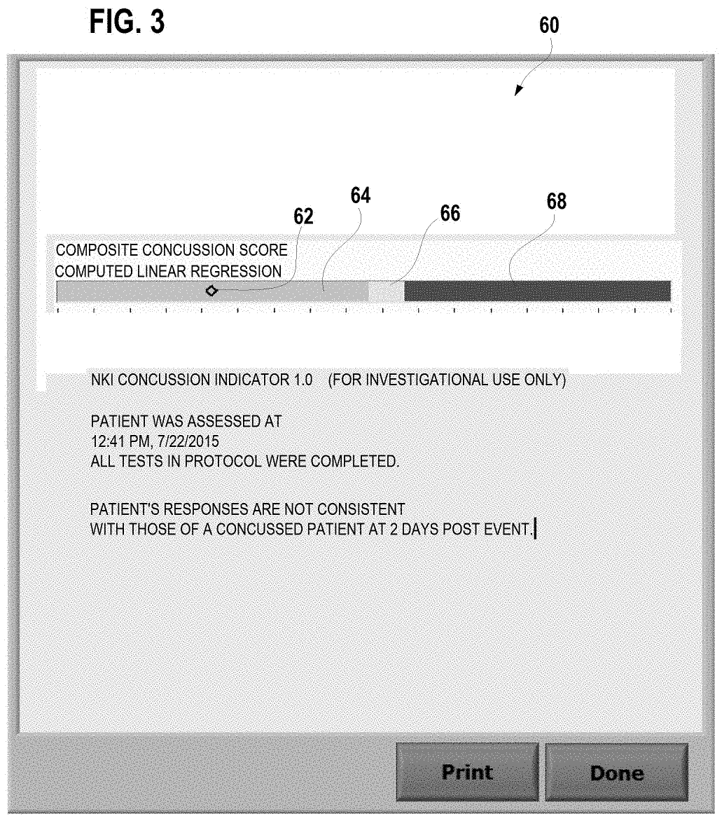

The present invention creates final interface for displaying test results. As shown in FIG. 3, the present invention creates a final user interface displaying the patient score 62 in reference to the distribution of concussion patient and control subject scores. The proposed score will be a value between 0 and 1, and will be presented within a spectrum of green 64—representing high probability of control or normal result, yellow 66—representing modest probability of concussion or other designated condition and red 68—representing a high probability of concussion or other designated condition. In similar existing OVRT technologies, the operator must examine each output graph individually to identify and record abnormal and normal measures of interest. In the invention, the several measurements upon which the scoring algorithm will be based are extracted, weighted and displayed automatically. This automated analysis is possible in the proposed medium following the implementation of the above aspects of the invention, necessary to reduce variability and noise. By automating pupil detection and removal of remaining artifacts, acquired data are to have sufficient reliability to minimize, possibly obviate, the need for operator filtering and extraction.

The invention may further guide the user through the process of conducting the concussion test battery. Upon completing the battery, the user interface may present the output of the proposed scoring mechanism. It is anticipated that the software may contain: On-screen scripts for instructions to patient; Weighting of measurements and calculation of score; User interface to record test progress; and User interface to display final results

Classification Model Example

As discussed above the present invention can be used with a variety of OVRT test models. The present invention may be effectively implemented with the following representative modeling that executes the following battery of OVRT tests:

Test Battery for NOTC Composite Concussion Score Prototype

| | | Est. |
|---|---|---|
| | | Test Time |
| Tests | Notes | (seconds) |
| Calibration | Pursuit type stimulus dot | 48 |
| Spontaneous & | 3 sec on and 10 seconds | 30 |
| gaze horizontal | off fixation, test for | |
| | nystagmus | |
| Predictive saccade | 20 saccades | 20 |
| Horizontal random | 30 saccades | 55 |
| saccade | | |
| Vertical random | 30 saccades | 55 |
| saccade | | |
| Smooth pursuit | 0.1 Hz and 0.75 Hz at 10 | 46 |
| horizontal | deg displacement | |
| Smooth pursuit | 0.1 Hz and 0.75 Hz at 10 | 46 |
| vertical | deg displacement | |
| Anti-saccade | 16 Anti-saccades | 90 |
| Optokinetic | 20 and 60 d/s | 70 |
| Visual reaction time | 20 cycles | 66 |
| Saccade and reaction | 30 cycles | 66 |
| time | | |
| Auditory reaction | 20 cycles | 66 |
| time | | |
| Sinusoidal harmonic | 0.02 Hz and .64 Hz | 120 |
| acceleration | | |
| Visual enhancement | 0.64 Hz | 20 |
| Visual suppression | 0.64 Hz | 20 |
| Subjective visual | 4 repetitions | 20 |
| vertical | | |
| Subjective visual | 4 repetitions | 20 |
| horizontal | | |
| crHIT | Acceleration 1,000 d/s², 10 cycles | 150 |

It is noteworthy that in the analysis of the physiologic parameters measured in the above testing, 5 to 10 variables (Key Variables) have been identified as having a significant contribution to classification accuracy (the greater the number of key variables the greater the accuracy of the testing, with highly diminishing increases in accuracy following 10 key variables). To date, each “Key Variable” identified has been generated from a single test, implying that only 5 to 10 tests will be included in the final concussion score protocol. Hence, it is anticipated that the per subject testing time for a final optimized Composite Concussion Score test battery will not exceed 15 minutes (and possibly far less time). As noted in this example the methodology uses oculomotor, vestibular, and reaction time (OVRT) testing on a on an NOTC, a rotational chair/oculomotor testing system, as a concussion assessment tool prototype. This model yields a reliable and concise method for performing concussion diagnosis and assessment.

There may be a perceived or a potential risk or deficiency with the present invention, namely that OVRT testing may not identify concussions that do not specifically impinge upon the vestibular, oculomotor, and hand-movement brain networks directly responsible for these functions, e.g. concussions with primarily cognitive or memory components, or concussion patients presenting with symptoms of “fogginess” or fatigue. The present invention minimizes this risk as the present invention targets and addresses this issue in three ways: i) the inclusion and exclusion criteria is designed to include all manifestations of concussion; ii) the clinically investigated battery of tests have included cognitive, memory, reaction time, and perceptual components and taxing what are thought to be important sets of neural pathways that can be disrupted by concussions; and iii) this extensive test battery, by its nature, assesses the functioning of a broad range of neuro-anatomical sites.

It may be beneficial to review the Cognitive, memory, reaction time, sensory components of proposed tests. Several tests in the representative modeling test battery protocol test and expose deficits in these areas. The visual and auditory reaction time tests assess both perception and reaction time. The oculomotor tests tax visual sensory function, including the perception of movement.

Some tests have cognitive and memory components, such as the Anti-saccade Test (AS): Test that requires subjects to look away from suddenly-appearing stationary targets, thereby suppressing and counter-acting the natural tendency to look toward visually salient events. The AS test has been employed in many types of studies assessing cognitive and executive function. A plausible hypothesis for the impact of the AS test is that deficits in short term memory (i.e. failing to remember the instruction to look away, not toward) and cognition can be detected via measurements of latency, accuracy, and percentage of pro-saccadic error. In a preliminary analysis of NKI's modeling data a logistic regression identified the AS pro-saccadic error percentage as a top five variable contributing to concussion classification.

ii) Saccade and Visual Reaction Time Test (SVRT): Test—30 visual targets appear in pseudo-random horizontal positions and are each stationary for a pseudo-random time (˜1-2 seconds). The subject is instructed to look to each target and press either a left or right button to record whether the stimulus was projected to the right or to the left of the previous position. The SVRT requires the subject to recall the previous position of the stimulus, determine the relative nature of the movement, then correctly signal the direction of movement. As with the AS test, the SVRT test has been identified as a significant variable in the logistic regressions performed on the preliminary modeling data; iii) Predictive Saccades Test (PS): Test—subject is directed to follow a dot as it is displayed. The subject is presented with 6 pseudo-randomly timed and positioned saccade targets, followed by 20 mirrored (same) saccade stimuli with fixed displacement and onset, i.e. target is displayed “back and forth” between two repeated positions at a fixed time interval. The PS test presents subjects with a “hidden” pattern measuring their ability to identify the pattern, and anticipate the timed movements. Subjects without impairment identify and begin predicting after only a few stimuli. Although a specific neural mechanism for this phenomenon is not proposed here, the test clearly assesses something beyond the vestibular, oculomotor, and manual reaction time systems.

A discussion of the “Neuroanatomical diversity in concussion” may be benefitial. Concussion pathology manifests as a broad spectrum of potential injury sites with a comparably broad presentation of clinical symptoms. The primary advantage of a diverse battery of tests comprising the test methodology, and using these as a base to optimize model development, is that good performance on these tests is dependent upon the integrity of a vast range of brain areas. Put another way, the test battery used to develop the model casts a very broad net anatomically, meaning that nearly any injury would impinge on the integrity of one or more areas that the tests reflect. This allows the invention to uncover the optimal test battery that can detect the disruptions caused by the many machinations of concussions. This underlying hypothesis, namely that oculomotor testing (especially in combination with vestibular, reaction time, visual, and oculomotor-based cognitive testing) can detect pathologies that are not specifically oculomotor or vestibular is supported by anatomical, physiological, and imaging studies that show that visual and oculomotor pathways extend through most major axonal fiber bundles and connect a wide range of brain regions. Diffuse axonal injury is present in concussion in major projections such as the corpus callosum, the cerebellar peduncles, and the anterior corona radiata. The test battery taxes all primary visual pathways (thalamus and V1), the motion-sensitive visual areas (e.g. area MT), the primary auditory pathway into the temporal lobe, decision-making areas (e.g. LIP), pre-central motor areas (e.g. FEF, SEF), cognitive and frontal lobe areas, as well as significant portions of the basal ganglia, both the medial and lateral cerebellum, and a multitude of brainstem areas (e.g. the superior colliculus, auditory nuclei, vestibular nuclei, oculomotor nuclei).

There is another perceived or potential risk related to the present invention, namely that regardless of the method's assessment capabilities, a technology capable of performing the tests may not be readily adopted due to the practical requirements and complexity of a rotational chair. There are two parts to addressing this perceived problem, the first is that the NOTC composite concussion score of the present invention, and other motion based nystagmography technologies like it, are extensive neurological assessment platforms. Such an extensive test battery as in the examples of this invention exhibit the sensitivity and specificity superior to currently available concussion assessment tools. While a rotary chair nystagmograph platform is not fieldable in the sense that it cannot be applied outside of hospital environments, it provides both a viable option for advanced, stationary clinical settings and, the detailed tracing of these 19 tests can be used by an MD specialist to observe specific abnormal responses, not just the concussion composite score. Secondly it is apparent that Key aspects of the proposed methodology are not specific to NOTC type systems. A portable assessment system (I-Portal® PAS, or I-PAS) that employs a battery of OVRT tests that will consists only of a head-mounted goggle-based display and eye tracking unit, controlled by a portable computer (a laptop). While without a rotational vestibular component, the I-PAS technology does include other features not available on rotary chairs, is easily fieldable, and early data collection and analysis suggest that the I-PAS test battery will also successfully identify concussions.

Secondary Test Battery Example

| TEST | mTBI Study Protocol | Example measures |
|---|---|---|
| Spontaneous | 1 cycle - Stimulus light | Amount, rate, and |
| nystagmus | is projected at a central | direction of |
| | fixation point for 3 | spontaneous nystagmus |
| | seconds, followed by light | |
| | off for 15 seconds. | |
| Optokinetic | 2 cycles - Full field random | Integrity of fixation |
| nystagmus | dot stimulus continually | reflex combining |
| | moves left 10 seconds, and | pursuits and saccades |
| | then right 10 seconds, at | |
| | 20°/second and 60°/second | |
| | velocity | |
| Smooth | Single light stimulus moves | Gain of eye relative to |
| pursuit - | smoothly left, then right, | stimulus; presence of |
| horizontal | with sinusoidal velocity and | saccadic intrusions |
| | maximum displacement of 10°. | |
| | 3 cycles at 0.1 Hz, 5 cycles at | |
| | 1.0 Hz, 7 cycles at 1.25 Hz. | |
| Smooth | Same as horizontal, but with | Same |
| pursuit - | 5 cycles at 0.75 Hz, 7 cycle | |
| vertical | s at 1.0 Hz. | |
| Saccade - | 30 cycles - Single light | Saccade onset latency, |
| random | stimulus projected at random | accuracy, presence of |
| horizontal | horizontal displacements and | corrective saccades |
| | time, with maximum | |
| | displacement of 30°, and | |
| | time between stimuli 1.2 to | |
| | 2.0 seconds. | |
| Saccade - | Same as horizontal, but with | Same |
| random | maximal vertical displacement | |
| vertical | of 20°. | |
| Saccade - | 20 cycles - | Ability to adapt to |
| predictive | Single light stimulus is | predictable timing and |
| horizontal | projected at 10° left or | position (latency and |
| | right displacement (alternating) | accuracy of saccades) |
| | with a fixed 0.65 second | |
| | interval. | |
| Saccade - | Same as horizontal, using 10° | Same |
| predictive | up and down displacements. | |
| vertical | | |
| Saccade - | Same as Saccade - random | Number of incorrect |
| antisaccade | horizontal, except that subject | pro-saccades, corrective |
| horizontal | is instructed to look away from | anti-saccades; latency |
| | the target. | |
| Saccade and | Single light stimulus | Saccade onset latency, |
| reaction time | projected at random horizontal | accuracy, presence of |
| | displacements and time, with | corrective saccades, |
| | maximum displacement of 30°, | latency and S.D. for |
| | and time between stimuli 1.2 to | left and right buttons |
| | 2.0 sec, subject asked to click | |
| | left or right buttons depends | |
| | on direction of saccades. | |
| Visual | 20 cycles, random single light | Latency and latency |
| Reaction | stimulus appears at center of | standard deviation |
| time | vision and subject using his/ | |
| | her dominate hand click on | |
| | button on | |
| Auditory | 20 cycles, random auditory | Latency and latency |
| reaction time | stimulus 85 decibel presented | standard deviation |
| | and subject using his/her | |
| | dominate hand click on button | |
| | as stimulus on | |
| Subjective | 6 cycles - Straight line | Angular error from |
| visual - | stimulus appears tilted off | vertical axis |
| vertical | vertical axis, up to 30° | |
| | displacement clockwise or | |
| | counterclockwise. Subject | |
| | asked to press buttons to | |
| | tilt line back to vertical | |
| | alignment. | |
| Subjective | 6 cycles - Same as vertical, | Angular error from |
| visual - | except asked to tilt line | horizontal axis |
| horizontal | until it is horizontal. | |

The present invention is not limited to concussion models, the present invention is applicable for OVRT testing protocols for other specific neuro-otologic conditions. For example, Internuclear ophthalmoplegia (INO) is a disorder of conjugate lateral gaze in which the affected eye shows impairment of adduction. The disorder is caused by injury or dysfunction in the medial longitudinal fasciculus (MLF), a heavily-myelinated tract that allows conjugate eye movement. In young patients with bilateral INO, Multiple Sclerosis often the cause. In older patients with one-sided lesions a stroke is a distinct possibility. However, there is a long list of possible causes. Currently, audiologists are diagnosing INO through direct observation of the patient or of a recording of the patient (i.e. a trace of eye position). A suitable OVRT testing protocol for diagnosing INO is based upon these existing tests and can be used with the present invention.

Ocular Lateral Pulsion (OLP) is caused by infarcts in the distribution of the posterior inferior cerebellar artery or distribution of the superior cerebellar artery. With regard to the diagnosis of OLP the objective saccade eye movement of a goggle based VOG system provide an objective tool for diagnosis of OLP thus a suitable OVRT testing protocol which includes several saccade inducing tests for diagnosing OLP used with the present invention is available.

Glissades eye movements are when eye velocity slows just prior to reaching the eye target and the eye gradually acquires the target or steps with a small additional saccade. Glissades eye movements can be caused by a cerebellar disorder, eye muscle or nerve weakness or head movement during the test. Current diagnosis is through visual inspection of patient eye response. With regard to the diagnosis of glissades eye movement the objective saccade eye movement of a goggle based VOG system provide an objective tool for diagnosis of glissades eye movement and a suitable OVRT testing protocol for diagnosing glissade eye movements may be used with the present invention.

Additionally the measurement and display of secondary, and higher, corrective saccades may be used in suitable OVRT testing protocols used with the present invention for Progressive Supernuclear Palsy (PSP) and other degenerative cerebellar disorders that cause highly saccadic results. Progressive supranuclear palsy (PSP) is a rare brain disorder that causes serious and permanent problems with control of gait and balance. The most obvious sign of the disease is an inability to aim the eyes properly, which occurs because of lesions in the area of the brain that coordinates eye movements. Some patients describe this effect as a blurring. PSP patients often show alterations of mood and behavior, including depression and apathy as well as progressive mild dementia. Initial complaints in PSP are typically vague and an early diagnosis is always difficult. PSP is often misdiagnosed because some of its symptoms are very much like those of Parkinson's disease, Alzheimer's disease, and rarer neurodegenerative disorders, such as Creutzfeldt-Jakob disease. In fact, PSP is most often misdiagnosed as Parkinson's disease early in the course of the illness. Memory problems and personality changes may also lead a physician to mistake PSP for depression, or even attribute symptoms to some form of dementia. The key to diagnosing PSP is identifying early gait instability and difficulty moving the eyes, the hallmark of the disease, as well as ruling out other similar disorders, some of which are treatable. Thus the present invention can greatly improve proper and early PSP diagnosis.

Other common conditions for which OVRT protocols used with the present invention are possible include Positional vertigo; Meniere's disease; Vestibular neuritis; Migraine-associated dizziness; Superior canal dehiscence; Vestibular schwannoma; and generally other central nervous system or general medical causes of imbalance and dizziness.

It is understood, therefore, that this invention is not limited to the particular embodiments disclosed, but it is intended to cover modifications that are within the spirit and scope of the invention, as defined by the appended claims and equivalents thereto. The preferred embodiments described above are illustrative of the present invention and not restrictive hereof. It will be obvious that various changes may be made to the present invention without departing from the spirit and scope of the present invention. The precise scope of the present invention is defined by the appended claims and equivalents thereto.

1.-20. (canceled)

21. A noninvasive rapid screening of neuro-otologic condition of a subject using combination of a subject's objective oculomotor, vestibular and reaction time analytic variables, comprising the steps of:
Establishing an oculomotor, vestibular and reaction time testing protocol for a specific neuro-otologic condition which protocol includes eye tracking aspects and which protocol obtains a combination of a subject's objective oculomotor, vestibular and reaction time analytic variables from a camera based eye tracker capable of capturing and quantifying eye movement velocity data, eye movement latency data, eye movement smoothness data and eye movement precision data;
Coupling the camera based eye tracker to the subject;
Automatically establishing a region of interest for the camera based eye tracker in which the region of interest is centered on the subject's pupils;
Conducting the oculomotor, vestibular and reaction time testing protocol to obtain a combination of a subject's objective oculomotor, vestibular and reaction time analytic variables based upon the quantified camera based eye tracker obtained data;
Analyzing the combination of a subject's objective oculomotor, vestibular and reaction time analytic variables obtained from camera based eye tracker to calculate a composite test protocol score for the subject; and
Displaying the composite test protocol score for screening of a neuro-otologic condition of the subject.

22. The noninvasive rapid screening of neuro-otologic condition of a subject according to claim 21 wherein the conducting the oculomotor, vestibular and reaction time testing protocol uses pre-programmed instructions to the subject.

23. The noninvasive rapid screening of neuro-otologic condition of a subject according to claim 22, further including the step of confirming that the oculomotor, vestibular and reaction time testing protocol is performed within preset parameters including automated eye image evaluation and advising a clinician of testing which is outside of present parameters.

24. The noninvasive rapid screening of neuro-otologic condition of a subject according to claim 23, wherein the displaying of the composite test protocol score is on graphical progressive background.

25. The noninvasive rapid screening of neuro-otologic condition of a subject according to claim 24 wherein the displaying of the composite test protocol score is a display of a normalized test score.

26. The noninvasive rapid screening of neuro-otologic condition of a subject according to claim 25 wherein the specific neuro-otologic condition is mild traumatic brain injury.

27. The noninvasive rapid screening of neuro-otologic condition of a subject according to claim 21, wherein the specific neuro-otologic condition is mild traumatic brain injury.

28. The noninvasive rapid screening of neuro-otologic condition of a subject according to claim 27, wherein the displaying of the composite test protocol score is on graphical progressive background.

29. The noninvasive rapid screening of neuro-otologic condition of a subject according to claim 28 wherein the displaying of the composite test protocol score is a display of a normalized test score.

30. The noninvasive rapid screening of neuro-otologic condition of a subject according to claim 29, further including the step of confirming that the oculomotor, vestibular and reaction time testing protocol is performed within preset parameters including automated eye image evaluation and advising a clinician of testing which is outside of present parameters.

31. The noninvasive rapid screening of neuro-otologic condition of a subject according to claim 21, further including the step of confirming that the oculomotor, vestibular and reaction time testing protocol is performed within preset parameters including automated eye image evaluation and advising a clinician of testing which is outside of present parameters.

32. The noninvasive rapid screening of neuro-otologic condition of a subject according to claim 31, wherein the displaying of the composite test protocol score is on graphical progressive background.

33. The noninvasive rapid screening of neuro-otologic condition of a subject according to claim 32 wherein the displaying of the composite test protocol score is a display of a normalized test score.

34. The noninvasive rapid screening of neuro-otologic condition of a subject according to claim 33 wherein the specific neuro-otologic condition is mild traumatic brain injury.

35. Automated data acquisition, appraisal and analysis in noninvasive rapid screening of neuro-otologic condition using combination of subject's objective oculomotor, vestibular and reaction time analytic variables obtained by a camera based eye tracker, comprising the steps of:
Establishing an oculomotor, vestibular and reaction time testing protocol for a specific neuro-otologic condition which protocol includes eye tracking aspects and which protocol obtains a combination of a subject's objective oculomotor, vestibular and reaction time analytic variables from a camera based eye tracker capable of capturing and quantifying eye movement velocity data, eye movement latency data, eye movement smoothness data and eye movement precision data;
Coupling the camera based eye tracker in a goggle based system to the subject;
Automatically establishing a region of interest for the camera based eye tracker in the goggle based system in which the region of interest is centered on the subject's pupils;
Conducting the oculomotor, vestibular and reaction time testing protocol to obtain a combination of a subject's objective oculomotor, vestibular and reaction time analytic variables based upon the quantified camera based eye tracker obtained data;
Analyzing the combination of a subject's objective oculomotor, vestibular and reaction time analytic variables obtained from camera based eye tracker to calculate a composite test protocol score for the subject; and
Displaying the composite test protocol score for screening of a neuro-otologic condition of the subject.

36. The automated data acquisition, appraisal and analysis according to claim 35 further comprising the step of automatically establishing a region of interest for the eye tracker and wherein the region of interest is centered on the subject's pupils.

37. The automated data acquisition, appraisal and analysis according to claim 35 wherein the specific neuro-otologic condition is mild traumatic brain injury.

38. The automated data acquisition, appraisal and analysis according to claim 35, wherein the displaying of the composite test protocol score is on graphical progressive background and wherein the displaying of the composite test protocol score is a display of a normalized test score.

39. The automated data acquisition, appraisal and analysis according to claim 15, further including the step of confirming that the oculomotor, vestibular and reaction time testing protocol is performed within preset parameters including automated eye image evaluation and advising a clinician of testing which is outside of present parameters.
